(19) United States
(12) Patent Application Publication
(10) Pub. No.: US 2010/0010349 A1
(43) Pub. Date: Jan. 14, 2010

(54) Image Depth Setting in an Ultrasound System

(75) Inventors: Sung In Park, Seoul (KR); Eun Ho Yang, Seoul (KR); Jung Sik Song, Seoul (KR); Su Myeong Lee, Seoul (KR); Soo Hwan Shin, Seoul (KR)

Correspondence Address: JONES DAY, 222 EAST 41ST ST, NEW YORK, NY, 10017, US

(73) Assignee: Medison Co., Ltd.

(21) Appl. No.: 12/499,059

(22) Filed: Jul. 7, 2009

(30) Foreign Application Priority Data
Jul. 10, 2008 (KR) 10-2008-0066780

(51) Int. Cl. A61B 8/14

(52) U.S. Cl. 600/443

(57) ABSTRACT

Embodiments for setting an image depth in an ultrasound system are disclosed. In one embodiment, a storage unit stores a mapping table of image depth information and gestation information. A user input unit receives input information from a user. A control unit retrieves from the storage unit image depth information corresponding to the input information and sets an image depth based on the retrieved image depth information.

The present application claims priority from Korean Patent Application No. 10-2008-0066780 filed on Jul. 10, 2008, the entire subject matter of which is incorporated herein by reference.

### TECHNICAL FIELD

The present disclosure generally relates to an ultrasound system, and more particularly to image depth setting in an ultrasound system.

### BACKGROUND

Recently, an ultrasound system has been extensively used in the medical field due to its non-invasive and non-destructive nature. Modern high-performance ultrasound systems and techniques are commonly used to produce two-dimensional and three-dimensional ultrasound images of internal features of a target object.

Generally, the ultrasound system transmits/receives ultrasound signals to/from the target object, thereby outputting electrical receive signals. Also, the ultrasound system forms an ultrasound image of the target object by using the receive signals depending on an image depth, which represents the range of an ultrasound image of the target object to be formed in a depth direction.

### SUMMARY

Embodiments for image depth setting are disclosed herein. In one embodiment, by way of non-limiting example, an ultrasound system comprises: a storage unit for storing a mapping table of image depth information and gestation information; a user input unit configured to receive input information from a user: and a control unit configured to retrieve from the storage unit image depth information corresponding to the input information, the control unit being further configured to set an image depth based on the retrieved image depth information.

In another embodiment, there is provided a method of setting an image depth in an ultrasound system, comprising: a) receiving input information from a user input unit within the ultrasound system; b) retrieving image depth information corresponding to the input information from a storage unit within the ultrasound system; and c) setting an image depth based on the retrieved image depth information by using a control unit within the ultrasound system.

In yet another embodiment, there is provided a computer readable medium comprising computer executable instructions configured to perform the following acts: receiving input information from a user; retrieving image depth information corresponding to the input information; and setting an image depth based on the retrieved image depth information.

The Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a flowchart showing a procedure of setting an image depth.

### DETAILED DESCRIPTION

A detailed description may be provided with reference to the accompanying drawing. One of ordinary skill in the art may realize that the following description is illustrative only and is not in any way limiting. Other embodiments of the present invention may readily suggest themselves to such skilled persons having the benefit of this disclosure.

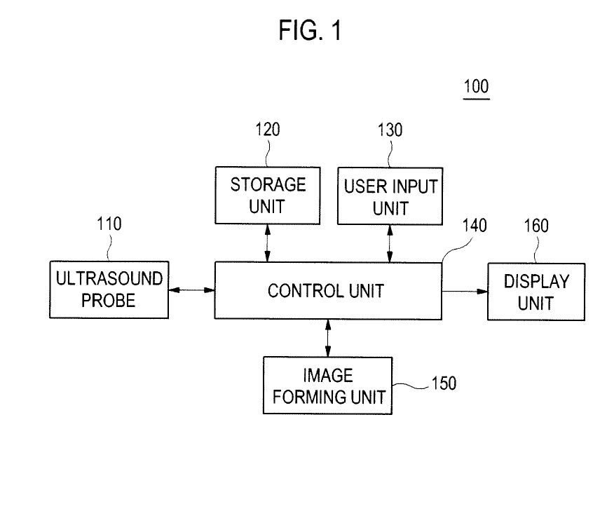

Referring to FIG. 1, an ultrasound system 100 in accordance with an illustrative embodiment is shown. As depicted therein, the ultrasound system 100 may include an ultrasound probe 110 configured to transmit/receive ultrasound signals to/from a fetus in a target object, thereby outputting received signals. In one embodiment, the ultrasound probe 110 may include an endocavity probe and a convex probe. However, the ultrasound probe 110 may not be limited thereto.

The ultrasound system 100 may include a storage unit 120 configured to store a mapping table of image depth information and gestation information. The image depth information may represent information upon a range of an ultrasound image of the target object to be formed in a depth direction. The gestation information may include gestational age information and last menstruation information. However, the gestation information may not be limited thereto. In one embodiment, the image depth information and the gestation information may be set depending on the type of the ultrasound probe. For example, the storage unit 120 may store the mapping table as shown in Table 1.

TABLE 1

| Ultrasound probe | Gestation information | Image depth information |
|---|---|---|
| Endocavity probe | 0~10 weeks | 8 cm |
| | 10~13 weeks | 10 cm |
| Convex probe | 0~12 weeks | 10 cm |
| | 12~20 weeks | 12 cm |
| | 20~30 weeks | 14 cm |
| | 30~40 weeks | 16 cm |

The ultrasound system 100 may include a user input unit 130 configured to receive input information from a user. The input information may include first input information for selecting gestation information. The input information may further include second input information for selecting an ultrasound probe among the endocavity probe, the convex probe and the like. However, the input information may not be limited thereto. The input unit 130 may be configured with a control panel, a mouse, a keyboard or the like capable of receiving the input information. However, the input unit 130 may not be limited thereto.

The ultrasound system 100 may include a control unit 140. The control unit 140 may be configured to set an image depth based on the mapping table and the input information.

The ultrasound system 100 may further include an image forming unit 150. The image forming unit 150 may be configured to form the ultrasound image of the fetus by using the received signals under the control of the control unit 140.

The ultrasound system 100 may further include a display unit 160 configured to the ultrasound image formed in the image forming unit 150. The display unit 160 may be one of a liquid crystal display, a cathode ray tube, a plate panel display and the like, which are capable of displaying the ultrasound. However, the display unit 160 may not be limited thereto.

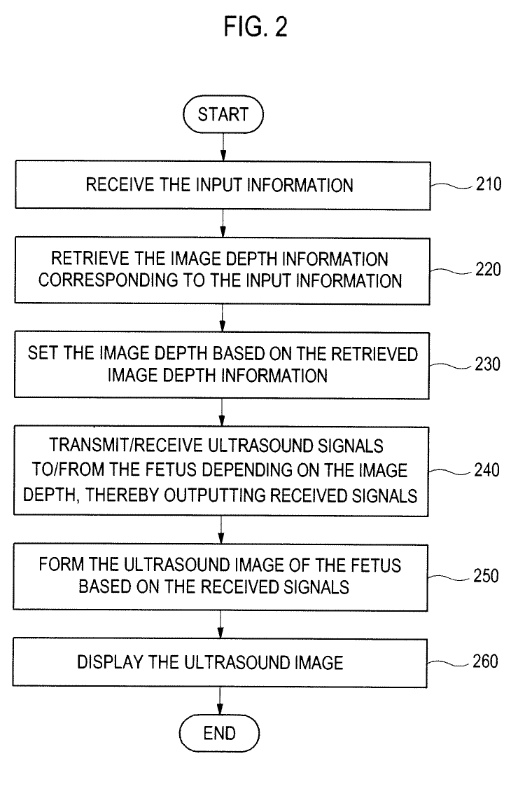

FIG. 2 is a flowchart showing a procedure of setting an image depth. Referring to FIG. 2, the user input unit 130 may receive from a user the input information at block 210.

The control unit 140 may retrieve from the storage unit 120 the image depth information corresponding to the input information at block 220. In one embodiment, the control unit 140 may analyze the input information. If the first input information comprises last menstruation information, the control unit 140 may calculate a gestational age based on the last menstruation information, and retrieve from the storage unit 120 the image depth information corresponding to the calculated gestational age and the input information. Otherwise, if the first input information comprises gestational age information, the control unit 140 may retrieve from the storage unit 120 the image depth information corresponding to the input information.

The control unit 140 may set the image depth based on the retrieved image depth information at block 230. The ultrasound probe 110 may transmit/receive the ultrasound signals to/from the fetus depending on the image depth under the control of the control unit 140, thereby outputting received signals at block 240.

The image forming unit 150 may form the ultrasound image of the fetus by using the received signals under the control of the control unit 140 at block 250. The display unit 160 may display the ultrasound image formed in the image form unit 150 at block 260.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
a storage unit for storing a mapping table of image depth information and gestation information;
a user input unit configured to receive input information from a user; and
a control unit configured to retrieve image depth information corresponding to the input information from the storage unit, the control unit being further configured to set an image depth based on the retrieved image depth information.

2. The ultrasound system of claim 1, wherein the gestation information comprises gestational age information and last menstruation information.

3. The ultrasound system of claim 2, wherein the input information includes first input information for selecting gestation information.

4. The ultrasound system of claim 3, wherein the control unit is configured to analyze the input information, if the input information comprises the last menstruation information, then calculate a gestational age based on the last menstruation information and retrieve the image depth information corresponding to the calculated gestational age from the storage unit.

5. The ultrasound system of claim 1, further comprising:
an ultrasound probe configured to transmit/receive ultrasound signals to/from a fetus in a target object to output received signals depending on the image depth.

6. The ultrasound system of claim 5, further comprising:
an image forming unit configured to form an ultrasound image of the fetus by using the received signals.

7. The ultrasound system of claim 5, wherein the image depth information and gestation information are set depending on a kind of the ultrasound probe.

8. The ultrasound system of claim 7, wherein the input information includes first input information for selecting gestation information and second input information for selecting the ultrasound probe.

9. A method of setting an image depth in an ultrasound system, comprising:
a) receiving input information from a user input unit within the ultrasound system;
b) retrieving image depth information corresponding to the input information from a storage unit within the ultrasound system; and
c) setting an image depth based on the retrieved image depth information by using a control unit within the ultrasound system.

10. The method of claim 9, wherein the storage unit stores a mapping table of image depth information and gestation information.

11. The method of claim 10, wherein the gestation information comprises gestational age information and last menstruation information.

12. The method of claim 11, wherein the input information includes first input information for selecting gestation information.

13. The method of claim 12, wherein the step b) comprises:
analyzing the input information;
if the input information comprises the last menstruation information, then calculating a gestational age based on the last menstruation information; and
retrieving from the storage unit the image depth information corresponding to the calculated gestational age.

14. The method of claim 9, further comprising:
transmitting/receiving by using an ultrasound probe within the ultrasound system ultrasound signals to/from a fetus in a target object to output received signals depending on the image depth.

15. The method of claim 14, further comprising:
forming by using an image forming unit within the ultrasound system an ultrasound image of the fetus by using the received signals.

16. The method of claim 9, wherein the image depth information and gestation information are set depending on a type of an ultrasound probe.

17. The method of claim 16, wherein the input information includes first input information for selecting gestation information and second input information for selecting the ultrasound probe.

18. A computer readable medium comprising computer executable instructions configured to perform following acts:
receiving input information from a user;
retrieving image depth information corresponding to the input information; and
setting an image depth based on the retrieved image depth information.

19. The computer readable medium of claim 18, wherein the computer executable instructions are further configured to perform a following act:
transmitting/receiving ultrasound signals to/from a fetus in a target object to output received signals depending on the image depth.

20. The method of claim 19, wherein the computer executable instructions are further configured to perform a following act:
forming an ultrasound image of the fetus by using the received signals.
